(19) United States
(12) Patent Application Publication
(10) Pub. No.: US 2026/0232885 A1
(43) Pub. Date: Aug. 13, 2026

(54) Ambulatory System with ECMO Device

(71) Applicant: UNIVERSITEIT TWENTE, Enschede (NL)

(72) Inventors: Quint Meinders, Enschede (NL); Danny Johannes Menno Van Galen, Enschede (NL); Jutta Arens, Enschede (NL)

(21) Appl. No.: 19/153,450

(22) PCT Filed: Feb. 5, 2024
(86) PCT No.: PCT/NL2024/050056

§ 371 Date: Aug. 4, 2025

(30) Foreign Application Priority Data
Feb. 6, 2023 (NL) 2034096

(51) Int. Cl. A61M 1/36; A61M 1/16

(52) U.S. Cl. CPC A61M 1/3603; A61M 1/1601; A61M 1/1629; A61M 1/1698; A61M 1/3623; A61M 1/3659; A61M 1/3666; A61M 2202/0007; A61M 2202/0014; A61M 2202/0021; A61M 2202/0208; A61M 2202/0225; A61M 2202/0413; A61M 2205/3331; A61M 2205/3368; A61M 2205/366; A61M 2205/8237; A61M 2209/086; A61M 2209/088; A61M 2210/06; A61M 2210/083; A61M 2210/086; A61M 2210/10; A61M 2210/12; A61M 2230/04

(57) ABSTRACT

A system comprises an ambulatory system comprising an extracorporeal membrane oxygenation system, a mobile gas supply, and a mobile frame. The oxygenation system comprises (i) a rechargeable power supply, (ii) an oxygenator, and (iii) a heat exchanger for controlling temperature of blood flowing through the oxygenation system. The system operates in an ambulatory or stationary mode. In the ambulatory mode (i) the temperature control fluid inlet and outlet are closed, (ii) the power supply provides the power for the extracorporeal membrane oxygenation system, and (iii) the gas supply is fluidically coupled to the oxygenator. In the stationary mode (i) a stationary temperature control fluid supply is coupled to the fluid inlet and a stationary temperature control fluid discharge is coupled to the temperature control fluid outlet, (ii) a stationary power supply is coupled to the oxygenation system, and (iii) a stationary gas supply is fluidically coupled to the oxygenator.

### FIELD OF THE INVENTION

The invention relates to a system to provide life support.

### BACKGROUND OF THE INVENTION

WO2020163596 A1 describes a transportable extracorporeal system includes a housing, a blood flow inlet, a blood flow outlet, a plurality of hollow gas permeable fibers, a gas inlet in fluid connection with inlets of the plurality of hollow gas permeable fibers, a gas outlet in fluid connection with outlets of the plurality of hollow gas permeable fibers, a first moving element, a concentrated oxygen generating device, a second moving element, a hollow transport conduit having a proximal opening and a distal opening and a power source configured to provide power to the first and second moving elements. The plurality of hollow gas permeable fibers comprising a gas transfer membrane. The concentrated oxygen generating device is configured to recycle waste oxygen from the gas transfer membrane to increase throughput and remove, by an adsorption/desorption process, unwanted gasses.

EP3220973 describes an oxygen supply unit for use with a blood oxygenator comprising an oxygen concentrator and a carbon dioxide scrubber. In an on-line operational mode, oxygen-rich gas from the oxygen concentrator is predominantly supplied to the blood oxygenator with a reduced flow of recycled gas from the concentrator. In an off-line operational mode where the oxygen supply unit is being powered by battery only, a larger flow of recycled gas from the blood oxygenator is passed through the carbon dioxide scrubber and combined with a lesser amount of oxygen-rich gas from the oxygen concentrator. The oxygen supply unit may be used in combination with a blood pump and oxygenator to provide ambulatory blood oxygenation to patients with compromised lung function.

### SUMMARY OF THE INVENTION

Extracorporeal Membrane Oxygenation (ECMO) is a temporary lifesaving treatment for critically ill patients suffering from severe respiratory and/or cardiac failure. Studies have demonstrated the feasibility of mobilization in ECMO patients within the hospital during their bridge to recovery or transplantation. Ambulatory ECMO is a comprehensive form of mobilization aimed at preventing neuromuscular weakness and impaired physical functioning during and after treatment.

In extracorporeal membrane oxygenation (ECMO), blood is pumped outside of a human body to a system that removes carbon dioxide and provides oxygen-rich (oxygenated) blood back to the body. ECMO is used in critical care situations, for instance for people having a very bad lung condition. ECMO may further be used during surgery to bypass the lungs and optionally the heart. Nowadays ECMO is also used in care to support people suffering from critical COVID-19, ARDS, and other infections. Whereas these people were initially left to rest to recover, it was lately discovered that having them exercise, such as letting them move (walk) facilitates the healing process.

Despite more compact and mobile ECMO devices, the implementation of ambulatory ECMO remains a labour-intensive, complex, and challenging operation. Ambulatory ECMO requires a large multidisciplinary team to carry all the equipment, to monitor and physically support the patient, and to provide a back-up wheelchair in case of patient fatigue. Additionally, current device configuration contributes to unnecessary device transport. Moreover, there is no adequate solution for ensuring the stability of the patient's cannula and circuit management during ambulation.

Common solutions to have the person walk is supporting the person with a large number of caregivers. A large number of caregivers (e.g., at least four or five) is required to prevent the person from falling as well as to carry parts of the medical equipment. Furthermore, patients may also need to be transported from one location (such as a hospital bed) to another location, such as an operation room. Hence, (also) for such transport a large number of caregivers is required.

Further, mobile ECMO systems are known. These systems may be flexible and less heavy, so less caregivers may be required to support the moving person using the system. However, most of these systems may only be used for lying transport, but not for mobilizing a patient as they are lacking a walking aid, an emergency seat, and a secure connection of the tubing and the cannula. Alternatively, mobile systems are known, e.g., as described in EP3220973, for relatively fit patients who do not require caregivers to support them during walking and who may even carry (parts of) the system themselves, e.g., in a wearable case. Furthermore, known solutions appear not to be usable for patients of intensive care units.

Hence, there is a need to combine the flexibility of a mobile ECMO system and the robustness of the more general static ECMO systems.

Hence, it is an aspect of the invention to provide a system to provide life support, which preferably further at least partly obviates one or more of above-described drawbacks. The present invention may have as object to overcome or ameliorate at least one of the disadvantages of the prior art, or to provide a useful alternative.

Hence, in a first aspect the invention provides a system. The system especially is a system to provide life support. In specific embodiments, the system comprises an ambulatory system. In embodiments, the ambulatory system may comprise an extracorporeal membrane oxygenation (or “ECMO”) system. Further, in embodiments, the ambulatory system may comprise a mobile gas supply. Furthermore, in embodiments, the ambulatory system may comprise a mobile frame. In embodiments, the extracorporeal membrane oxygenation system (herein further also indicated as the “ECMO system”) may comprise a rechargeable power supply. The ECMO system may in further embodiments comprise an oxygenator. In further specific embodiments, the ECMO system may further (also) comprise a heat exchanger. Especially, the heat exchanger may facilitate controlling a temperature of blood flowing through the extracorporeal membrane oxygenation system. In further embodiments, the heat exchanger may facilitate (especially depending on a mode of the system) controlling a temperature of the blood flowing through the extracorporeal membrane oxygenation system, especially through the oxygenator and especially also through a first side (or “first compartment”) of the heat exchanger. The heat exchanger is described in more detail further below. Furthermore, in embodiments, the heat exchanger, especially a second side of the heat exchanger, may comprise a temperature control fluid inlet and a temperature control fluid outlet. Especially, the heat exchanger may comprise a temperature control fluid inlet and a temperature control fluid outlet at a second side (or “second compartment”) of the heat exchanger (thermally conductively coupled to the first side of the heat exchanger)). In embodiments, the system may be configured to operate in one or more modes. The one or more modes may especially comprise one or more of an ambulatory mode and a stationary mode. In the stationary mode, the ECMO system may be connected to a stationary power supply. Further, in the stationary mode, the ECMO system may further be coupled to a stationary heater-cooler. The ECMO system may in further embodiments in the stationary mode be coupled to a stationary gas supply. In the stationary mode, the extracorporeal membrane oxygenation system may especially be connected to one or more of a stationary power supply, a stationary heater-cooler, and a stationary gas supply. In embodiments, in the stationary mode, a stationary temperature control fluid supply may be (releasably) coupled to the temperature control fluid inlet and further especially a stationary temperature control fluid discharge may be (releasably) coupled to the temperature control fluid outlet (of the heat exchanger). Especially, in specific embodiments, the temperature control fluid inlet and the stationary temperature control fluid supply may be in open fluid connection and especially (also) the temperature control fluid outlet and the stationary temperature control fluid discharge may be in open fluid connection. Further, in the stationary mode the stationary power supply may especially be (releasably) coupled to the extracorporeal membrane oxygenation system. In embodiments, the stationary power supply may provide the power for (or to) the extracorporeal membrane oxygenation system. Especially, the stationary power supply may provide the power to power one or more power consuming elements (see further below) of (or comprised by) the extracorporeal membrane oxygenation system. Furthermore, in the stationary mode the stationary gas supply may be fluidically coupled to the oxygenator, especially for providing an oxygen containing fluid to the blood flowing through the oxygenator. The stationary gas supply may especially provide (or supply) the oxygen containing fluid, especially an oxygen containing gas. In the ambulatory mode, the extracorporeal membrane oxygenation system may especially be connected to the rechargeable power supply. In further embodiments, the extracorporeal membrane oxygenation system may be connected to the mobile gas supply, in the ambulatory mode. Furthermore, in the ambulatory mode, one or more, especially all of the temperature control fluid inlet and the temperature control fluid outlet (of the heat exchanger) may especially be closed. In specific embodiments, the rechargeable power supply may provide the power for (or to) the extracorporeal membrane oxygenation system (when connected to the ECMO system) (in the ambulatory mode). The rechargeable power supply may provide the power, especially to power one or more power consuming elements of the extracorporeal membrane oxygenation system. In further specific embodiments, the mobile gas supply may be fluidically coupled to the oxygenator for providing an oxygen containing fluid to the blood flowing through the oxygenator (in the ambulatory mode).

Here, the terms “releasably coupled” and “releasably connected”, such as in the phrase “the stationary temperature control fluid supply is (releasably) coupled to the temperature control fluid inlet” and comparable phrases especially refer to a coupling or connection between a system (or an element) and another system or element (especially in a first mode), wherein the connection is further configured to allow decoupling (or releasing) the systems and/or elements from each other, i.e. the system (or the element) may have the functionality of both being coupled to another system (or element) and not being coupled to another system (or element). That is, a releasably coupled system (or element) may be either in a coupled mode or a released mode, where in the coupled mode, the releasably coupled system may be configured coupled to another system (or element), and in the released mode, the releasably coupled system (or element) may not be coupled to the other system (or element). Herein, sometimes only the terms “connected” or “coupled” are used to refer to releasably coupled (connected). It will be clear from the context if such connection may be decoupled (during use). The terms “coupled” and “connected”, and comparable terms may be used interchangeably.

Further, the term “fluidically coupled” may refer to a system (or an element) which may be configured to be in fluidic connection with another system or element, wherein a fluid may be flowed from the system (or element) to the other system (or element), especially through a closed conduit without any (substantial) loss of the fluid.

In a specific embodiment, the invention provides a system (to provide life support,) comprising an ambulatory system, the ambulatory system comprising an extracorporeal membrane oxygenation system, a mobile gas supply, and a mobile frame, wherein the extracorporeal membrane oxygenation system comprises (i) a rechargeable power supply, (ii) an oxygenator, and (iii) a heat exchanger, especially for controlling a temperature of blood flowing through the extracorporeal membrane oxygenation system, wherein the heat exchanger comprises a temperature control fluid inlet and a temperature control fluid outlet, wherein the system is configured for operating in a stationary mode and for operating in an ambulatory mode, wherein in the stationary mode the extracorporeal membrane oxygenation system is connected to (i) a stationary power supply, (ii) a stationary heater-cooler, and (iii) a stationary gas supply, and wherein in the ambulatory mode (i) the extracorporeal membrane oxygenation system is connected to the rechargeable power supply and to the mobile gas supply and (ii) the temperature control fluid inlet and the temperature control fluid outlet are closed.

In a further specific embodiment, the invention provides a system (to provide life support,) comprising an ambulatory system, the ambulatory system comprising an extracorporeal membrane oxygenation system, a mobile gas supply, and a mobile frame, wherein the extracorporeal membrane oxygenation system comprises (i) a rechargeable power supply, (ii) an oxygenator, and (iii) a heat exchanger for controlling a temperature of blood flowing through the extracorporeal membrane oxygenation system, wherein the heat exchanger comprises a temperature control fluid inlet and a temperature control fluid outlet, wherein the system is configured for operating in an ambulatory mode and for operating in a stationary mode; wherein in the ambulatory mode: (i) the temperature control fluid inlet and the temperature control fluid outlet are closed, (ii) the rechargeable power supply provides the power for the extracorporeal membrane oxygenation system, and (iii) the mobile gas supply is fluidically coupled to the oxygenator; and wherein in the stationary mode: (i) a stationary temperature control fluid supply is coupled to the temperature control fluid inlet and a stationary temperature control fluid discharge is coupled to the temperature control fluid outlet, (ii) a stationary power supply is coupled to the extracorporeal membrane oxygenation system, wherein the stationary power supply provides the power to the extracorporeal membrane oxygenation system, and (iii) a stationary gas supply is fluidically coupled to the oxygenator.

As mentioned above, the system may be used to support a person or patient whose lungs may not be able to provide enough oxygen to the blood of the person. The system is especially a mobile system allowing the person to be moved with the system (while being connected to the extracorporeal membrane oxygenation system. The system may further be used if the person is immobile and e.g., laying in a bed.

Such a system provides the benefit that the system may be moved (along) with the patient from the immobile situation to an ambulant situation without disconnecting the system from the person. The system may be arranged next to a patient in a (hospital) bed for supporting the patient with their respiratory (or lung) functioning. The system may function optimally in the stationary as well as in the ambulatory situation with the required respective functions and using the resources required. For instance, the system is in embodiments especially configured to heat up the blood of the patient when the patient is in the stationary situation (in rest), whereas the blood will be heated by the patient when the patient moves in the ambulatory situation (wherein the heater may be disconnected). If the patient has to be moved to another location, the system may continue supporting the person because the system and the patient may be moved jointly. Further, embodiment of the system may allow the patient to be ambulant while keeping the required support. Moreover, the system may also support the person physically while being ambulant. This way, the number of nurses, doctors or other caregivers that are required to support a patient with an ECMO system while moving from a first location to another location may drastically reduce. The system especially requires less staff members that may be needed using prior art system to carry all the equipment during ambulation. Currently, a team of 5 to 6 caregivers may be required to ambulate ECMO patients. With the system of the invention this may be reduced to 1 to 2 caregivers. In embodiments, e.g., a single person may support the patient while being moved (or while moving). The system may facilitate moving (walking or exercising) of the patient (in an easy way). The system may especially advantageously be used in an intensive care unit (“ICU”).

Embodiments of the system may further simplify the transition between the ambulatory mode and the stationary mode, allowing a fast switch. Furthermore, the system is especially configured such that elements of the system that have to be worn/carried by the patient are minimized. Furter in embodiments, the system is very versatile. Moreover, most standard prior art ECMO systems may be introduced in the system. The system not necessary comprises a dedicated ECMO system. The invention is especially directed to the configuration of connections between the ECMO system and the patient and configurations of connections between elements of the ECMO system and/or further elements of the system as well as configurations of connections between the ECMO system and features that are already standard present near a bed of the patient, such as a power supply and a (external) gas supply.

As mentioned above, the invention especially provides a system to provide life support comprising an ambulatory (or mobile) system. The ambulatory system especially comprises the ECMO system. The term “ECMO system” or “extracorporeal membrane oxygenation system” especially refers to a system comprising an artificial lung or a heart-lung machine. With the system, blood of a patient may be oxygenated outside the body instead of via the lungs. When (being) connected to the ECMO system, blood flows through a tubing to an artificial lung or “oxygenator” of the ECMO system that adds oxygen, takes out carbon dioxide, and then flows back into the body. Further, the system may be configured to warm the blood to body temperature, such as in the range of 33-41° C., especially 35-39° C. More especially, the system may be configured to warm the blood to a temperature in the range of 36-38° C., especially 36.5-37.5° C. The ECMO system may especially comprise an ECMO device or ECMO machine. Moreover, the ECMO device or ECMO machine may in embodiments be a standard ECMO machine or device known in the art. The system may for instance be configured for connecting to a known ECMO machine (device), such as the device in EP3220973.

The term “oxygenator” especially relates to an artificial lung. The oxygenator is configured for providing oxygen to blood, flowing through the oxygenator, and especially also for removing carbon dioxide from the blood. The oxygenator may provide a gas exchange. The oxygenator may especially comprise a membrane. The membrane is especially permeable to oxygen and carbon dioxide and impermeable to the blood. The blood may be provided at a first side of the membrane and a gas supply may be provided at the other side of the membrane. The blood may especially flow through a fluid circuit and may be flowed along the membrane, and may e.g., be returned to a patient. The (oxygen containing) gas provided to the other side not necessary is returned and may in embodiments (after the gas exchange) leave the oxygenator to the atmosphere. The oxygenator may thus comprise an inlet (a fluid inlet) and an outlet (a fluid outlet) for connecting (the blood) with (a space at) the first side of the membrane. The oxygenator may further comprise an inlet (a gas inlet) for connecting the gas supply with (a space at) the other side of the membrane, and especially an outlet (a gas outlet) for releasing gas (from the space) again at the other side of the membrane.

In specific embodiments, the ambulatory system comprises the mobile frame. In embodiments, the mobile frame may (be configured to) support (or “carry”) one or more elements comprised by the system, for example the ECMO system (or parts of the ECMO system). The mobile frame may (further) (be configured to) support the mobile gas supply. In further embodiments, the mobile system may (also) (be configured to) support the rechargeable power supply and/or the oxygenator and/or the heat exchanger. In yet further embodiments, the mobile frame may further (be configured to) carry a pump (see further below). The mobile frame is especially configured to support (carry) substantially all (required) system elements in the ambulatory mode. In the ambulatory mode, especially the patient substantially does not need to carry hardware of the system, and may only be connected to the system, especially the ECMO system (via tubing), wearing a brace or a body support.

Hence, in embodiments, the mobile frame may (be configured to) carry (support) one or more of, especially all of (when comprised by the system) the ECMO system (especially including an integrated or separately configured rechargeable power supply, oxygenator, heat exchanger, and fluid pump), a mobile gas supply, a gas splitter, part of the tubing, and an medical element (see below). The frame is especially configured to relieve the patient from carrying parts of the system. Moreover, also the staff may be relieved by the configuration of the mobile frame. The frame especially requires less staff members that are normally needed to carry all the equipment during ambulation.

In specific embodiments, the extracorporeal membrane oxygenation system is physically secured to the mobile frame. In further embodiments, the ECMO system may be supported by the mobile frame. The ECMO system may be carried by the mobile frame, and in embodiments(configured) around it (preventing the ECMO system to fall off the mobile frame). In embodiments, the ECMO system may be physically secured to the mobile frame by means of screws, bolts, nails, pins or other fasteners. Further, in embodiments, the ECMO system may be physically secured to the mobile frame by means of straps. Furthermore, in embodiments, the ECMO system may be physically secured to the mobile frame by means of fixtures, wherein the fixtures may comprise male-female connectors. Yet further, in embodiments, the mobile frame may comprise rails for sliding the ECMO system onto the mobile frame, thus physically securing the ECMO system to the mobile frame.

The term support (or carried) in phrases like “the frame (or another element) supports the element” especially refers to supporting/carrying such that the element will be moved when moving the mobile frame (or the other element). The element may in embodiments be indirectly carried and not directly contact the mobile frame. For instance, the heat exchanger may be carried by the mobile frame if the heat exchanger is carried by the ECMO system, and the ECMO system is carried by the mobile frame. The term may further include that the element will not fall of the mobile frame, e.g., because it is enclosed by the mobile frame, or it is (physically) secured to the mobile frame.

Here, the term “physically secured” may refer to a system (or an element) in physical contact with another system or element such that there is no (or only a minimal) relative motion between the two systems (or elements).

Hence, in embodiments, the mobile frame supports the mobile gas supply. Especially, the mobile gas supply may be a supply of gas, wherein the mobile gas supply may provide gas to the ECMO system, especially to the oxygenator. In embodiments, the mobile gas supply may provide a gas comprising oxygen (“oxygen comprising gas”). The mobile gas supply may in embodiments comprise one or more of a gas tank (with a flow regulator), a pony bottle, an oxygen concentrating device, etc. Note that, in embodiments, gas may be provided to the ECMO system from an external gas supply, such embodiments are discussed further below. The mobile gas supply may provide the benefit of providing gas to the ECMO system when the ECMO system is in the ambulatory mode and is not connected to an external gas supply. Hence, in this way, gas (of the mobile gas supply) may especially be provided to the ECMO system when the ECMO system is in motion. For example, the ECMO system (carried by and/or physically secured to the mobile frame) may be moved with a patient and may still be in operation.

In embodiments, the ECMO system comprises the rechargeable power supply, the oxygenator, and the heat exchanger. The ECMO system may especially be powered either by the rechargeable power supply comprised by the ECMO system, or by an external power supply (such embodiments are discussed further below). The rechargeable power supply may provide the benefit of providing power (or electricity) to the ECMO system when the ECMO system is in the ambulatory mode and is not connected to the external power supply. The power may be provided to a power consuming element arranged at or on the frame (especially supported by the mobile frame), such as a pump (see below), the ECMO system, or for instance an oxygen concentration device. Hence, in this way, power may especially be provided to the ECMO system when the ECMO system is in motion (such as in the ambulatory mode). For example, (in the ambulatory mode) the ECMO system (supported by the mobile frame) may be moved with a patient and may still be in operation.

In embodiments, the oxygenator may be configured for (i) removing carbon dioxide from the blood and providing oxygen to the blood flowing through the oxygenator. The oxygenator may especially comprise a membrane separating two fluids wherein gas may permeate through the membrane from one of the fluid to the other one of the fluids (see also above). Especially, oxygen may permeate to (and diffuse into) the blood and carbon dioxide may (diffuse out of) and permeate away from the blood. In embodiments, the blood may be flowed through a fluidic circuit, wherein the oxygenator is configured along the fluidic circuit. Hence, the blood may be oxygenated as it is flowed through the fluidic circuit. Features of the fluidic circuit are discussed further below.

In embodiments, the heat exchanger may be configured along the fluidic circuit. Hence, the heat exchanger may control the temperature of the blood flowing through the fluidic circuit. The heat exchanger may in embodiments comprise a first side (or “first compartment”) and a second side (or “second compartment”). In embodiments, the first side may be a part of the fluidic circuit. Therefore, in embodiments, the blood may be flowed through (a first compartment at) the first side of the heat exchanger. Further, in embodiments, the first side and the second side may be in thermal contact. I heat exchanger may comprise the temperature control fluid inlet and the temperature control fluid outlet. In embodiments, a fluid supply may be provided to the temperature control fluid inlet (at the first side) and a fluid discharge may be received from the temperature control fluid outlet (at the first side). Hence, in this way a second fluid may be flowed through (a second compartment at) the second side of the heat exchanger. Especially, the heat exchanger may control the temperature of the first side by controlling the temperature of the second side. Therefore, the blood flowing through the first side may be heated (or cooled) by the heat exchanger. Hence, the blood flowing through the ECMO system may especially be heated by the heat exchanger. Note that it may not be necessary to operate the heat exchanger at all instances. For example, the heat exchanger may provide the benefit of heating (or cooling) the blood when the patient is resting. If the patient is active/performing a physical exercise, such as walking in the ambulatory mode, heating of the blood may not be required. The fluid supply and the fluid discharge may in embodiments be releasably coupled, Hence, the heat exchanger may be decoupled from the fluid supply and the fluid discharge, such as when the ECMO system (comprising the heat exchanger) is moved. Hence, in this way, the ECMO system may in embodiments be operated even when mobile (such as in the ambulatory mode). This is particularly useful for moving the ECMO system along with the patient (in the ambulatory mode) such as when the patient has to be moved from location.

As mentioned above, the system may in embodiments be configured for operating in one or more modes, especially the ambulatory mode and the stationary mode. In embodiments, in the ambulatory mode the temperature control fluid inlet and the temperature control fluid outlet are closed. Note that the temperature control fluid may still remain within the heat exchanger, especially the temperature control fluid may remain on the second side of the heat exchanger. Hence, the heat exchanger may still provide heating (or cooling) for a short duration (for example 5 to 10 minutes) in the ambulatory mode. Furthermore, the system may be configured to operate in the ambulatory mode, wherein the ambulatory system may be moved along with the patient. In such a scenario, the movement (physical exercise) of the patient may (also) generate body heat. Hence, in the ambulatory mode, the additional heating (or cooling) of the blood may not (always) be necessary.

Further, in embodiments, in the ambulatory mode, the rechargeable power supply may provide the power to the ECMO system. Especially, the (ECMO) system may comprise (power consuming) elements that may (also) require to be powered. The ECMO system may especially comprise a (fluid) pump that may require to be powered. The (ECMO) system may in further embodiments comprise one or more solenoid valves that may require to be powered (see further below). Hence, in embodiments, the rechargeable power supply may provide power to the one or more power consuming elements of the extracorporeal membrane oxygenation system and/or of the system. In further specific embodiments, the system may comprise one or more further power consuming elements, e.g., a perfusion pump, or a monitor, that may also be provided with power from the rechargeable battery. In embodiments, the further power consuming is especially (also) carried by the mobile frame.

The term “rechargeable battery” may refer to a plurality of (different) rechargeable batteries. For instance, in embodiments a first rechargeable battery (such as a rechargeable battery integrated in the ECMO system) may provide the power to the ECMO system and a further rechargeable battery may be configured for providing power to a further power consuming element. Note that, in some embodiments, the ECMO system may comprise a dedicated rechargeable battery configured to power only the ECMO system, wherein the ECMO system may (also) comprise an additional rechargeable battery to power the one or more power consuming elements.

Furthermore, in embodiments, the mobile gas supply may be fluidically coupled to the oxygenator. As mentioned further above, the mobile gas supply may provide a gas comprising oxygen. In embodiments, the blood may be flowed through the oxygenator. Further, the oxygenator may especially provide oxygen to the blood and remove carbon dioxide from the blood.

In embodiments, the ambulatory system may comprise wheels for moving the ambulatory system, especially the mobile frame. Hence, in embodiments, the ECMO system and the mobile gas supply (supported by the frame) may especially be moved with the mobile frame. Hence, in the ambulatory mode, the mobile frame may be moved along with the patient and the ECMO system may be in operation while being moved.

In embodiments, the system may (also) be operated in a stationary mode. In embodiments, in the stationary mode the stationary temperature control fluid supply may be (releasably) coupled to the temperature control fluid inlet and a stationary temperature control fluid discharge may be (releasably) coupled to the temperature control fluid outlet. Especially, the temperature control fluid inlet and the stationary temperature control fluid supply may be in open fluid connection (in the stationary mode). Additionally, in embodiments, the temperature control fluid outlet and the stationary temperature control fluid discharge may be in open fluid connection (in the stationary mode). Hence, in this way, the blood flowing through the oxygenator may be heated (or cooled) in the stationary mode. The system may especially be configured for heating (or cooling) the blood to a (human) body temperature (especially in the range of 36.5-37.5° C.).

Further, in the stationary mode the stationary power supply may in embodiments be (releasably) coupled to the extracorporeal membrane oxygenation system. Especially, the stationary power supply may provide the power to the ECMO system. Furthermore, the stationary power supply may especially provide power to the one or more (additional or further) power consuming elements of the ECMO system. If the stationary power supply is coupled to the ECMO system, the mobile power supply may not provide the power to the ECMO system anymore. This may not necessarily imply that the mobile power supply is decoupled from the ECMO system. The mobile power supply may still be coupled to the ECMO system. Yet, an (electronic) system (e.g. a switch or a relay) may control that the power supply of the mobile power systems is stopped when the stationary system provides the power.

Furthermore, in the stationary mode the stationary gas supply may be fluidically coupled to the oxygenator for providing the oxygen containing fluid to the oxygenator. Especially the stationary gas supply may provide oxygen to the blood flowing through the oxygenator (in the stationary mode).

Additionally, in embodiments, the ambulatory system may be configured for (releasably) connecting to a stationary system comprising the stationary gas supply, a stationary heater-cooler and the stationary power supply. In embodiments, the ECMO system may be coupled to the stationary power supply (comprised by the stationary system). In further embodiments, the ECMO system, especially the oxygenator, may be fluidically coupled to the stationary gas supply (comprised by the stationary system). In yet further embodiments, the heater-cooler (comprised by the stationary system) may be fluidically coupled to the heat exchanger. The stationary system is discussed further below.

Hence, in this way, the ECMO system may be operated in a stationary mode when the patient is resting (for instance in a bed), wherein the ECMO system may be powered by the stationary power supply, the ECMO system may be fluidically coupled to the stationary gas supply, and the ECMO system may be releasably coupled to the stationary temperature control fluid supply and the releasably coupled to the stationary temperature control fluid discharge. Alternatively, the ECMO system may be operated in an ambulatory mode when the patient has to be moved from the first location to the second location, wherein the ECMO system is powered by rechargeable power supply and the ECMO system may be fluidically coupled to the mobile gas supply.

As mentioned above, the ECMO system may be releasably coupled to the mobile gas supply and the stationary gas supply. In embodiments, the ambulatory system may comprise a gas splitter or gas switching device. In specific embodiments, the gas splitter may be a three-way valve. In embodiments, the gas splitter may comprise a first (gas) inlet port, a second (gas) inlet port and a gas outlet port. Especially, a gas inlet of the oxygenator may be connected to the gas outlet port. In embodiments, the mobile gas supply may be connected to the first (gas) inlet port and the stationary gas supply may be connected to the second inlet port. Further, in embodiments, the gas splitter may be configured to fluidically connect (i) the first inlet port to a mobile gas supply outlet port of the mobile gas supply in the ambulatory mode and (ii) the second inlet to a stationary gas supply outlet port of the stationary gas supply in the stationary mode. In embodiments, the mobile gas supply may provide the gas supply via the mobile gas supply outlet port. Further, in embodiments, the stationary gas supply may provide the gas supply via the stationary gas outlet port.

Herein the terms “gas switching device” and “gas splitter” may be used interchangeably, especially referring to a device allowing to switch a supply of gas (or a fluid connection allowing a supply of the gas) to the oxygenator from the mobile gas supply to the stationary gas supply and vice versa (from the stationary gas supply to the mobile gas supply).

Note that in embodiments, the gas splitter may be toggled such that the mobile gas supply is either fluidically connected to the oxygenator or such that the stationary gas supply is fluidically coupled to the oxygenator. Further, in some embodiments, the gas splitter may be manually toggled such that oxygenator is in fluidic connection to the mobile gas supply or the stationary gas supply. However, in other embodiments, the gas splitter may be automatically toggled such that oxygenator is in fluidic connection to the mobile gas supply or the stationary gas supply. The automatic toggling of the gas splitter may in embodiments be facilitated mechanically wherein a pressure difference between the first (gas) inlet port and the second (gas) inlet port may determine the fluidic connection. Alternatively, in embodiments, the automatic toggling of the gas splitter may in embodiments be facilitated by an electronic system, such as comprising solenoids. The solenoids may open and close a (three-way) valve allowing to control which of the first (gas) inlet port and the second (gas) inlet port is in open fluidic connection with the gas outlet port). The solenoid may especially be configured such that the mobile gas supply is fluidically connected to the oxygenator if the solenoid is not actuated (powered). Hence, in embodiments (in the ambulatory mode), the mobile gas supply is fluidically coupled to the oxygenator via the gas switching device (gas splitter). In further embodiments (in the stationary mode), the stationary gas supply is fluidically coupled to the oxygenator via the gas switching device.

Furthermore, in embodiments, a mixture of gasses may be provided at the gas splitter outlet wherein the oxygenator is in fluidic connection with both the mobile gas supply and the stationary gas supply. Additionally, in embodiments, one or more mobile gas supplies, or one or more stationary gas supplies, or a combination thereof may be connected to the first inlet port and the second inlet port. In embodiments, the mobile gas supply and/or the stationary gas supply may comprise a flow regulator to control the supply of gas provided.

In embodiments, the stationary system may comprise one or more gaseous supplies selected from the group of a carbon dioxide-supply, a nitrogen supply, an oxygen supply, and an air supply fluidically coupled to a gas mixer for mixing the gaseous supplies to provide the gas supply. Hence, in this way, the composition of the gas supplied to the patient may especially be controlled.

In specific embodiments, the ambulatory system comprises a gas splitter, wherein the gas splitter comprises a first inlet port, a second inlet port and a gas outlet port, wherein the oxygenator is connected to the gas outlet port; wherein the mobile gas supply is connected to the first inlet port and the stationary gas supply is connected to the second inlet port; and the gas splitter is configured to fluidically connect (i) the first inlet port to the mobile gas supply in the ambulatory mode and (ii) the second inlet to the stationary gas supply in the stationary mode.

As mentioned above, the blood may be flowed through a fluidic circuit. A tubing may in embodiments be a part of the fluidic circuit, wherein the blood may be flowed via the tubing. In embodiments, the oxygenator and the heat exchanger (especially the first side of the heat exchanger) may be configured along the fluidic circuit (in series), especially the oxygenator and the heat exchanger may be configured along a first part of the fluidic circuit. In embodiments, the ECMO system may (additionally) comprise a fluid pump and the tubing. Especially, the fluid pump may pump the blood through the tubing. That is, the fluid pump may be configured for flowing the blood through the ECMO system. Note that, the oxygenator, the first side of the heat exchanger and the fluid pump may in embodiments be configured in series along the fluidic circuit. However, the order of the oxygenator, the heat exchanger and the fluid pump configured along the fluidic circuit may be different in different embodiments.

Furthermore, it may not especially be necessary for the fluid pump to pump the blood through the fluidic circuit. For instance, depending on the configuration of the ECMO system, the blood may be pumped by the patient's heart. In which case, the blood may be flowed via the fluid pump even when the fluid pump is configured off (or idle) (i.e., the blood is not pumped by the fluid pump). The different modes of operation of the ECMO system in relation to the patient are discussed further below.

In specific embodiments, the extracorporeal membrane oxygenation system comprises a fluid pump and tubing, wherein the fluid pump, the oxygenator, the heat exchanger and the tubing define a first part of a fluidic circuit for the blood, wherein the fluid pump is configured for flowing the blood through the extracorporeal membrane oxygenation system.

As mentioned above, the ambulatory system may be moved along with the patient. Especially, the patient may be functionally connected to the ECMO system. Especially, the (circulatory system of the) patient may define the second part of the fluidic circuit. Hence, the blood may be flowed via the first part of the fluidic circuit (wherein the blood may be heated (or cooled) and may be oxygenated) and via second part of the fluidic circuit (wherein the blood may flow through the blood vessels of the patient). In embodiments, the first part and the second part of the fluidic circuit may complete the fluidic circuit. Hence, in this way, the patient may be functionally connected to the ECMO system. Further hence, the ambulatory system may especially be moved with the patient functionally coupled to the patient.

In embodiments, the mobile frame may be configured to support a patient. The mobile frame may comprise one or more sides. Especially, the patient may be supported at a first side of the mobile frame. The sides of the mobile frame may in embodiments comprise frame elements such as bars, grills, handles, etc. Especially, the functional frame elements may facilitate the patient holding on to one or more functional frame elements and thus be supported by it. Additionally, or alternatively, the patient may stand on one or more functional frame elements and thereby support the patient. Furthermore, in embodiments, the mobile frame may (also) comprise (functional) frame elements that may facilitate the patient leaning on the mobile frame. Hence, in this way, the patient may especially walk along with the mobile frame and may be supported by the mobile frame. In further embodiments, the mobile frame may comprise frame elements that completely support the patient (for instance a frame element that may comprise a seat to support the patient). Such embodiments are discussed further below.

In embodiments, the mobile frame may comprise an adjustable frame element configured for extending from the first side (position) of the mobile frame, especially for supporting the patient. In embodiments, the adjustable frame element may be a part of the mobile frame that may be extended (for example an extendable rail or bar). Alternatively, or additionally, in embodiments, the adjustable frame element may be an element that may be folded against the frame. Note that the adjustable frame element may especially extend (away) from the frame, especially from the first side of the frame. Hence, in embodiments, the patient may be supported from one side wherein the mobile frame may support the patient at the first side (of the frame). In further embodiments, a first adjustable frame element may support the patient (in addition to the mobile frame supporting the patient at a first side).

In specific embodiments, the mobile frame is configured to support a patient at a first side of the mobile frame, wherein the mobile frame comprises an adjustable frame element configured for extending from the first side of the mobile frame for supporting the patient.

The term “adjustable frame element” may especially refer to a plurality of (different) frame elements. In embodiments, the mobile frame may comprise one or more adjustable frame elements at the first side of the mobile frame. In yet further embodiments, the mobile frame may comprise two adjustable frame elements configured to support the patient. In embodiments, the adjustable frame elements may be configured to support the patient from the lateral sides (of the patient) and the mobile frame may support the patient from the front side (of the patient). In specific embodiments, the adjustable frame element is configured for extending along one or two sides of the patient, for supporting the patient while walking.

In further embodiments, the adjustable frame element may comprise a seating support configured for supporting the patient while sitting. Especially, the patient may be supported by the seating support on at least one lateral side of the patient. In specific embodiments, the adjustable frame element comprises a seating support configured for supporting the patient while sitting. In further embodiments, the seating support may comprise a seating support element to provide a seat. Especially, the seating support element may be physically secured to the mobile frame. In embodiments, the seating support element may (also) be physically secured to the adjustable frame element. Hence, in this way, the patient may be seated and the ambulatory system may be configured to be moved with the patient (and while the ECMO system is in operation).

In a specific embodiment, the adjustable frame elements may be configured to support the patient from the lateral sides (of the patient) and the mobile frame may support the patient from the front side (of the patient), wherein the seating support may comprise a first seating support element (to support the patient while seated) and a second seating support element (to support the back of the patient). Hence, in embodiments, the one or more seating support elements may especially provide a mobile seat. Furthermore, the patient may be supported by the seating support, especially wherein the patient is movable (or mobile).

Hence, in specific embodiments, the seating support comprises a mobile seat configured for moving the patient in a seated position relative to a remainder of the frame.

The seating support may in embodiments be coupled to the mobile frame (i.e., the seating support is physically secured to the mobile frame) or the seating support may be decoupled from the mobile frame (i.e., the seating support is not physically secured to the mobile frame). Hence, the seating support may provide the benefit of transferring the patient from the seating support to a stationary chair or a bed with relative ease. Especially, the ambulatory system may be positioned in the vicinity of the bed and the patient may be transferred to the bed by decoupling the seating support. Especially, the patient may be seated facing the mobile frame and the posterior of the patient may be in the vicinity of a bed, and the patient may be transferable from the mobile seat to the bed by means of decoupling the seating support. Hence, in specific embodiments, at least part of the adjustable frame element is removable from the frame for moving the patient to and from the bed to a location on the first side of the frame.

As described above, the fluidic circuit may comprise the first part of the fluidic circuit and the second part of the fluidic circuit. Especially, the fluidic connection between the first part of the fluidic circuit and the second part of the fluidic circuit may be via a cannula. In embodiments, the cannula may be part of the first part of the fluidic circuit. Further, in embodiments, the cannula may comprise one or more cannula inserts (or lumens). The canula may especially be fluidically connected to the tubing of the first part of the fluidic system. In embodiments the tubing and the canula may be directly connected to each other. It will be understood that the tubing may also be fluidically coupled to the cannula via further tubing or piping arranged between the tubing and the canula (and extending the tubing). Herein it is further explained as the tubing is (directly fluidically connected to the cannula and yet, the tubing may comprise the further tubing and/or piping (connecting the tubing to the cannula).

In embodiments, the oxygenator may comprise an oxygenator inlet and an oxygenator outlet for circulating the blood. Further, in embodiments, the cannula may be configured for fluidically coupling to the oxygenator inlet and the oxygenator outlet of the oxygenator (via the tubing) for circulating the blood from the cannula through the oxygenator.

In embodiments, the fluidic circuit may especially be along a first cannula insert, the oxygenator inlet, the oxygenator outlet and a second cannula insert. Note that, in embodiments, the heat exchanger and/or a fluid pump may be configured along the fluidic circuit. Furthermore, in embodiments, the blood may flow via the patient's body (especially via the patient's circulatory system). Especially, the patient's circulatory system may be at least part of the second part of the fluidic circuit. Therefore, in this way, the patient may receive the oxygenated blood from the ECMO system.

In further embodiments, the system may comprise a body support. Especially, the body support may comprise a body wearable unit, and a locking plate. The body wearable unit is especially configured for fixating the tubing (for fluidically connecting the canula to ECMO system) to a person wearing the body support.

In embodiments, the cannula may be configured to be introduced to a patient, especially via injection. The cannula may especially be a tube (comprising an insert) that may be inserted into an artery, a vein, or a body cavity. The cannula may be introduced (or inserted) into the body of the patient and hence it may be advantageous to maintain the cannula physically secured to the body of the patient. It may further be advantageous to maintain the tubing physically secured to the body of the patient. Especially, it may be desired to arrest relative motion between the tubing and the location(s) of the body where the cannula may be inserted. Movement of the cannula at the location(s) of insertion of the body of the patient may especially cause damage to the body tissue. Further, the tubing may in embodiments be relatively large (such as the tubing may have an inner diameter of at least ⅛ in, such as at¼ast ¼ in, especially at least ⅜ in) and hence may require additional elements to physically secure the tubing to the patient's body (such that there is no relative motion between the cannula and the patient's body). Further, in embodiments, the tubing may have a diameter of at maximum 25 mm, at maximum 20, especially at maximum 15 mm.

In embodiments, the body wearable unit may contain straps, cables, buckles, etc. to physically secure the cannula (especially via the tubing) to the patient's body. The body wearable unit may especially be wrapped around an appendage or the body of a patient. In embodiments, the body wearable unit may be selected from the group of a shoulder strap, a head strap, a waist strap, a harness, a chest strap, an arm strap, and a leg strap.

As mentioned above, the body support may comprise a locking plate. Especially, the locking plate may be configured to physically secure the tubing (to the patient). Hence, in this way, the locking plate may physically secure the tubing such that there is no relative motion in the cannula tubing between the locking plate and the cannula. Especially, the locking plate may be configured for immobilizing the tubing. In embodiments, the locking plate may comprise one or more clasps, locking elements, fixtures, or jigs to physically secure the tubing to the locking plate. Hence, in this way, there may be no strain on the cannula tubing between the cannula and the locking plate. This provides the advantage that the remainder of the (cannula) tubing (excluding the cannula tubing between the cannula and the locking plate) may be moved around the patient without causing injury to the patient. Hence, the patient may especially be moved or be supported by the mobile frame while the ECMO system is still in operation. Further hence, this provides the benefit that the ECMO system may be (functionally) coupled to the patient without the requirement for additional caregivers to hold the tubing (including the cannula tubing) in place, even when the patient is sitting, standing, walking, or resting.

In specific embodiments, the system may comprise a body support comprising a body wearable unit and a locking plate, wherein the body wearable unit is selected from the group of a shoulder strap, a head strap, a waist strap, a harness, a chest strap, an arm strap and a leg strap, wherein the locking plate is configured for immobilizing a tubing fluidically connecting the ECMO system to a cannula.

In a further aspect, the invention may provide the body support described herein as such.

As mentioned above, the cannula may comprise one or more cannula inserts. Especially, the one or more cannula inserts (or lumens) may be configured to be inserted in an artery and/or a vein. The cannula may especially be inserted in a patient in one or more different configurations. In embodiments, a first configuration may be a veno-venous configuration. In the veno-venous configuration, the blood may be extracted from the vena cava and be returned to the vena cava, right atrium, or pulmonary artery. Such a configuration may support the respiratory functions of the patient by providing oxygen to the blood and thus, the patient. In such a configuration, the ECMO system may provide respiratory support, but the patient may still depend on their own heart to maintain blood circulation.

In embodiments, a second configuration may be a veno-arterial configuration. In the veno-arterial configuration, the blood may be extracted from the right atrium (or vena cava) and returned to an artery. In embodiments, such a configuration may bypass the lungs and the heart of the patient. Such a configuration may support the respiratory functions of the patient by providing oxygen to the blood and thus, the patient. Furthermore, in such a configuration, the ECMO system may further pump the blood through the fluidic circuit. In such a configuration, the ECMO system may provide respiratory and circulatory support.

In embodiments, a third configuration may be an arterio-venous configuration. The arterio-venous configuration may be analogous to the veno-arterial configuration, with the difference being the direction that the blood flows in the fluidic circuit. In embodiments, the blood may be extracted from an artery and returned to the right atrium. Such a configuration may support the respiratory functions of the patient by providing oxygen to the blood. Furthermore, in such a configuration, circulation may be maintained by the patient's heart. Hence, in such a configuration, the fluid pump may be configured off (or be absent).

The aforementioned configurations relate to the cannula comprising two inserts (or lumens). This may be referred to as dual-lumen cannulation. However, in embodiments, the cannula may (also) comprise three cannula inserts. This may be referred to as triple-lumen cannulation, resulting in veno-veno-arterial or veno-arterio-venous cannulation. The aforementioned triple-lumen cannulation may especially support the heart. Furthermore, the ECMO system may (additionally) support the respiratory functions in a triple lumen cannulation, referring to veno-pulmonary-arterial or veno-arterial-pulmonary arterial cannulation, respectively. Such types of cannulations are apparent to the skilled person.

In addition to the body support mentioned above, the (ambulatory) system may in embodiments comprise a (additional) medical element. Especially, the (additional) medical element may regulate and/or monitor the heart rate, the blood pressure, blood sugar levels, etc. Hence, in embodiments, the medical element may be selected from the group of an intra-venous drip, a heart rate monitor, and an emergency kit. Especially, the medical element may be functionally coupled to the mobile frame and other elements of the system.

The term “functionally coupled” especially indicates that the element such as the medical element may function when being connected. For instance, if the element requires power, the element may be coupled to a power supply. The term may refer to electrically coupled. Or, if the element requires to be in a fluidic connection with another element (to function), then the connection may (further) be a fluidical connection. The term may further refer to (being) fluidically coupled. Further, the functionally coupling may further indicate that the element is secured to the other element or system. The term electrically coupled such as in the phrase “the first element and the second element are electrically coupled” especially indicates that power may be provided by the first element to a second element or vice versa.

The mobile frame may especially (be configured to) support the medical element. In this way, the medical element may be moved with the ambulatory system. This may provide the benefit of providing care to the patient while the ambulatory system is moved along with the patient. Furthermore, such an ambulatory system may (also) provide care to the patient when the patient is resting (such as in the bed). Such a (ambulatory) system may further provide the advantage of being a single unit comprising the necessary elements to provide care to the patient. Yet further, the ambulatory system comprising the ECMO system and (additional) medical elements may be moved as a single unit along with the patient while (still) providing medical care to the patient.

In embodiments, the mobile frame may comprise a braking system. The ambulatory system may in embodiments be configured to provide (medical) care to the patient and hence, may be required to remain in proximity to the patient. Especially, since the patient may be functionally coupled to the ambulatory system, it may be desired that the ambulatory system remains stationary relative to the patient. Hence, the braking system may especially arrest the movement of the mobile frame. The mobile frame may be configured immobile by default (or “when being idle”). In further embodiments, the braking system may comprise a braking lever for unlocking the brake. The braking lever may comprise a lever (or handle) to pull or to push. Yet in further embodiments, the braking lever may be integrated in the frame and be actuated when holding the frame. Hence, the mobile frame may remain stationary until the braking lever is engaged. In specific embodiments, the mobile frame comprises a braking system, wherein the braking system comprises a braking lever, wherein the mobile frame is configured immobile by default.

In addition to the ambulatory system, the system may comprise a stationary system. In embodiments, the stationary system may comprise a heater-cooler, the stationary power supply, and the stationary gas supply.

In an operational mode, the heater-cooler may especially be fluidically coupled to the heat exchanger. In embodiments, the heater-cooler may comprise a heater-cooler inlet configured for coupling to the stationary temperature control fluid discharge. In further embodiments, the heater-cooler may comprise a heater-cooler outlet configured for coupling to the stationary temperature control fluid supply. For instance, in the stationary mode, the second side of the heat exchanger may be in fluidic connection with the heater-cooler. Especially, the heater-cooler may facilitate circulating a temperature control fluid via the second side of the heat exchanger. The heater-cooler may in embodiments comprise a heater-cooler pump to pump the temperature control fluid (through the heat exchanger). Thus, in this way, in the stationary mode, the temperature control fluid supply may be provided to the temperature control fluid inlet (of the heat exchanger) and the fluid discharge may be received from the temperature control fluid outlet. Further, in embodiments, the temperature control fluid flowing through the heater-cooler may be heated (or cooled). Hence, in this way, in the stationary mode, the heater-cooler may especially facilitate heating (or cooling) the heat exchanger (especially the first side of the heat exchanger).

The heater-cooler is especially used in the stationary mode. This way, the heater-cooler may not be part of, or comprised by, the ambulatory system. Moreover, in embodiments power is only consumed by the heater-cooler in the stationary mode, especially provided via the stationary power supply. In embodiments no power may be consumed (required) from the rechargeable power supply by the heater-cooler (since no heater-cooler is present in the ambulatory system.

In embodiments, the stationary system may comprise the stationary power supply for coupling to a static power line. That is, in embodiments, an external power supply may be electrically connected to the stationary power supply. Hence, the stationary power supply comprised by the stationary system may be a conduit for power (or electricity). In some embodiments, the stationary power supply (comprised by the stationary system) may be a power hub. Furthermore, in the stationary mode, the stationary power supply may provide the power to the ECMO system. Especially, in the stationary mode, the ECMO system may be electrically coupled to the static power line via the stationary power supply.

In embodiments, the stationary system may comprise the stationary gas supply for coupling to a static gas supply line. That is, in embodiments, an external gas supply may be fluidically connected to the stationary gas supply. Hence, the stationary gas supply comprised by the stationary system may be a conduit for one or more gases. In some embodiments, the stationary gas supply (comprised by the stationary system) may be a gas conduit. Furthermore, in the stationary mode, the stationary gas supply may provide the oxygen containing fluid (especially, the oxygen comprised gas) to the ECMO system, especially the oxygenator. Especially, in the stationary mode, the ECMO system may be fluidically coupled to the static gas line via the stationary gas supply.

Note that, in embodiments, the static power line may comprise a wall power supply. Furthermore, in embodiments, the one or more static gas supply lines may comprise a wall gas supply. In an environment such as a hospital, the stationary system may be configured in a room (or ward) and functionally coupled to both the wall power supply and the wall gas supply. Note that the stationary system is intended to remain in a room (or ward) for an extended duration of time, for example the duration for which the patient may be admitted to the room (or ward). Hence, in the stationary mode, the ambulatory system may be coupled to the stationary system as defined further above.

In specific embodiments, system further comprises a stationary system, wherein the stationary system comprises the heater-cooler comprising a heater-cooler inlet configured for coupling to the stationary temperature control fluid discharge and a heater-cooler outlet configured for coupling to the stationary temperature control fluid supply, the stationary power supply configured for coupling to a static power line, and the stationary gas supply configured for connecting to one or more static gas supply lines.

As mentioned above, in specific embodiments, in the stationary mode: (i) the power supply is coupled to the extracorporeal membrane oxygenation system, (ii) the stationary gas supply is coupled to the extracorporeal membrane oxygenation system, and (iii) the stationary temperature control fluid discharge is coupled to the temperature control fluid outlet and the stationary temperature control fluid supply is coupled to the temperature control fluid inlet.

In embodiments, the system may comprise a docking arrangement. The docking arrangement may especially facilitate the docking of the stationary system and the ambulatory system such that they are functionally coupled. Hence, in embodiments, the stationary system may comprise a stationary docking part. Further, in embodiments, the ambulatory arrangement may comprise an ambulatory docking part. Furthermore, the stationary docking part and the ambulatory docking part may be functionally coupled.

In embodiments, the stationary docking part may comprise a plurality of primary docking ports. Especially, the stationary power supply may be electrically coupled to a first primary docking port. The gas conduit may especially be fluidically coupled to a second primary docking port. In embodiments, the heater-cooler inlet may be coupled to a (first) third primary docking port. In further embodiments, the heater-cooler outlet may be coupled to a (second) third primary docking port.

Analogously, in embodiments, the ambulatory docking part may comprise a plurality of secondary docking ports. Especially, the ECMO system may be electrically coupled to a first secondary docking port. In embodiments, the oxygenator may be fluidically coupled to a second secondary docking port. The temperature control fluid outlet may in embodiments be fluidically coupled to a (first) third secondary docking port. In further embodiments, the temperature control fluid inlet may be fluidically coupled to a (second) third secondary docking port.

Hence, in the stationary mode, the stationary system may in embodiments be functionally coupled to the ambulatory system by functionally coupling the ambulatory docking part and stationary docking part. Especially, in the stationary mode, the first primary docking port may be electrically coupled to the first secondary docking port. Further, in embodiments, in the stationary mode, the second primary docking port may be fluidically coupled to the second secondary docking port. In further embodiments, in the stationary mode, the (first) third primary docking port may be fluidically coupled to the (first) third secondary docking port. Yet further, in the stationary mode, the (second) third primary docking port may be fluidically coupled to the (second) third secondary docking port. Hence, in this way, the ambulatory docking part and the stationary docking part may especially be functionally coupled. Note that the ambulatory system may in embodiments (also) be decoupled from the stationary system by decoupling the ambulatory docking part and the stationary docking part.

In specific embodiments, the system comprises a docking arrangement, wherein the docking arrangement comprises an ambulatory docking part and a stationary docking part, wherein the stationary docking part comprises a plurality of primary docking ports, wherein the stationary power supply is electrically coupled to a first primary docking port, wherein the gas conduit is fluidically coupled to a second primary docking port, wherein the heater-cooler inlet and the heater-cooler outlet are fluidically coupled to a third primary docking port, wherein the ambulatory docking part comprises a plurality of secondary docking ports, wherein the extracorporeal membrane oxygenation system is functionally coupled to the secondary docking ports, wherein in the stationary mode the ambulatory docking part and the stationary docking part are coupled, wherein the extracorporeal membrane oxygenation system is fluidically coupled to the stationary gas supply, wherein the extracorporeal membrane oxygenation system is coupled to the stationary power supply, and wherein the temperature control fluid inlet is coupled to the heater-cooler outlet and the temperature control fluid outlet is coupled to the heater-cooler inlet.

Note that the stationary system may especially be moved from a first location to a second location. For instance, when a patient is admitted to a room (or ward), the stationary system may be configured in the said room (or ward). Hence, in this way, the stationary system may remain in the vicinity of the patient when the patient is resting (such as in the vicinity of the bed), and the ambulatory system may be moved along with the patient, wherein the patient may be functionally coupled to the ambulatory system (when the ambulatory system is moved with the patient). Hence, the ambulatory system may be moved with the patient when the patient is moved from a first location to a second location, and the ambulatory system may be (functionally) coupled to the stationary system when the patient is resting (for example resting in the bed).

Hence, in embodiments, the stationary system may comprise (a set of) stationary system wheels for moving the stationary system. Hence, in this way, the stationary system may be moved from a first location to a second location. In embodiments, the ambulatory system and the stationary system may be physically coupled (even when the system is not operated in the stationary mode). This may provide the benefit of moving the stationary system along with the ambulatory system. For example, in a scenario where a series of tests have to be performed on the patient, the patient may have to be situated at an external location to the patient's room (or ward). In such a scenario, the stationary system may be moved to the external location along with the ambulatory system, especially the stationary system may be physically secured to the mobile frame (and moved with the mobile frame). Hence, in specific embodiments, the system may be configured for connecting the ambulatory system to the stationary system, wherein the stationary system is movable with the ambulatory system. Note further that in such a scenario, the patient may still be functionally coupled to the ECMO system wherein the ECMO system may operate in an ambulatory mode.

Hence, in embodiments, the mobile frame may further comprise a lift mechanism to lift the stationary system. Therefore, in an operational mode the stationary system may especially be lifted and physically secured to the mobile frame. Note that, in such an operational mode, the stationary system wheels may be disabled. Especially, the stationary system wheels may no longer be in contact with the ground. This may provide more stability to the mobile frame in moving both the ambulatory system and the stationary system (especially in instances wherein the stationary system wheels may not be compatible for moving with the mobile frame).

The embodiments described herein are not limited to a single aspect of the invention. For example, an embodiment describing the method may, for example, further relate to the system, especially to an operational mode of the system, or especially to the control system. Similarly, an embodiment of the system describing an operation of the system may further relate to embodiments of the method. In particular, an embodiment of the method describing an operation (of the system) may indicate that the system may, in embodiments, be configured for and/or be suitable for the operation. Similarly, an embodiment of the system describing actions of (a stage in) an operational mode may indicate that the method may, in embodiments, comprise those actions.

### BRIEF DESCRIPTION OF THE DRAWINGS

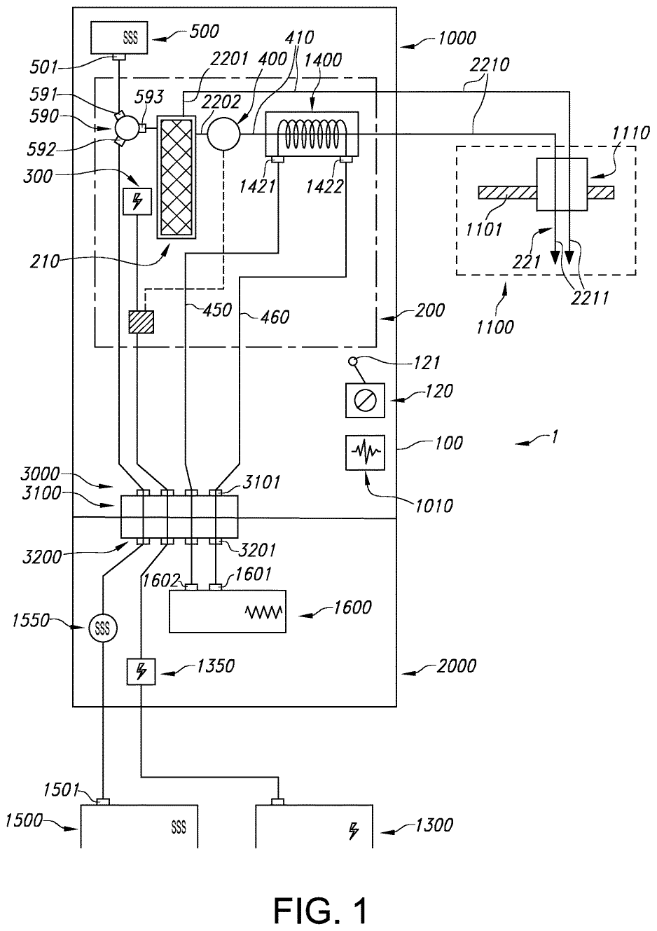

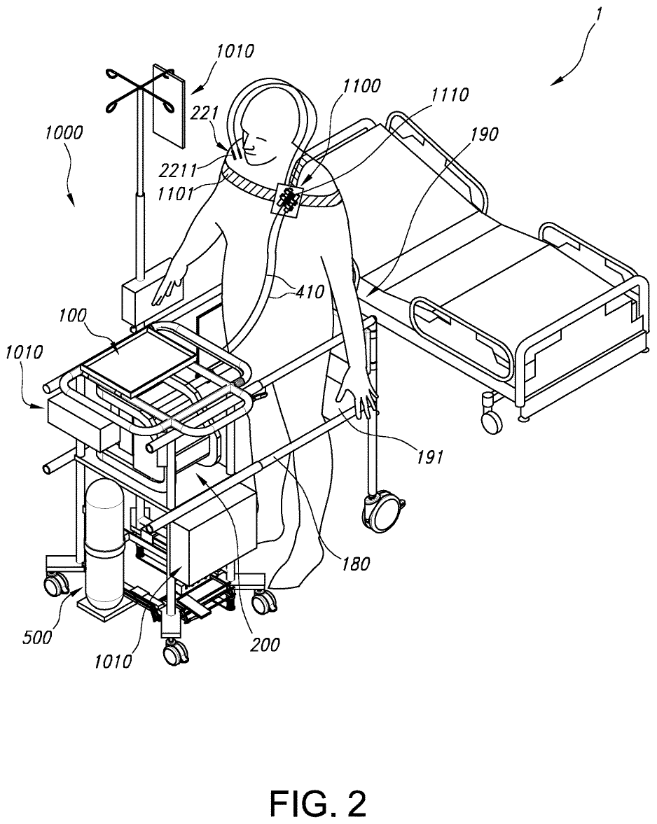

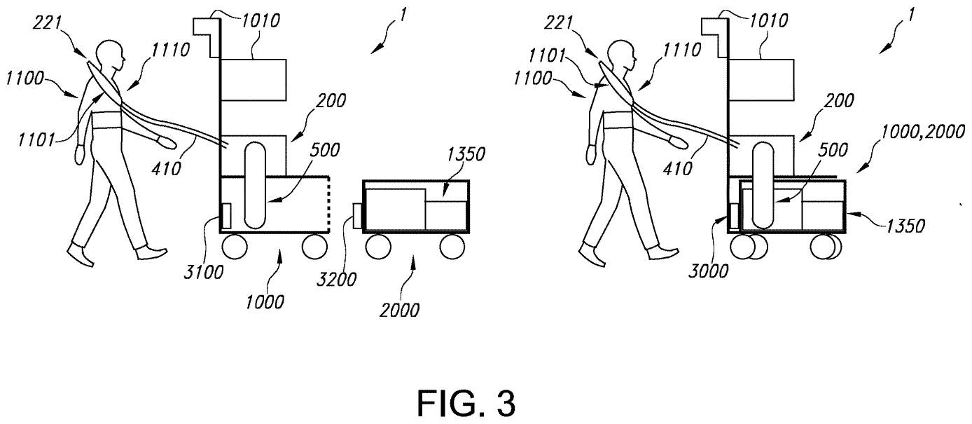

Embodiments of the invention will now be described, by way of example only, with reference to the accompanying schematic drawings in which corresponding reference symbols indicate corresponding parts, and in which: FIG. 1 schematically depicts an embodiment of the system, FIG. 2 schematically depicts the use of said system supporting a patient, and FIG. 3 depicts an embodiment of the system illustrating the docking of the ambulatory system and the stationary system. The schematic drawings are not necessarily to scale.

### DETAILED DESCRIPTION OF THE EMBODIMENTS

FIG. 1 schematically depicts an embodiment of the system 1. The invention provides a system 1 to provide lung and life support. The system 1 may comprise an ambulatory system 1000. In the depicted embodiment, the ambulatory system 1000 comprises an ECMO system 200, a mobile gas supply 500, and a mobile frame 100.

In embodiments, the ECMO system 200 may comprise the rechargeable power supply 300 to power the ECMO system 200. In embodiment, the rechargeable power supply 300 may comprise an integrated battery 300. In the depicted embodiment, the ECMO system 200 comprises (i) a rechargeable power supply 300, (ii) an oxygenator 210, and (iii) a heat exchanger 1400. As mentioned above, in some embodiments, the ECMO system 200 may have an integrated power supply 300 and an integrated heat exchanger 1400. However, in other embodiments, the ECMO system 200 may (also) separately comprise one or more of the rechargeable power supply 300 and the heat exchanger 1400. The heat exchanger 1400 may especially facilitate controlling a temperature of blood 2210 flowing through the ECMO system 200. In embodiments, the heat exchanger 1400 comprises a temperature control fluid inlet 1421 and a temperature control fluid outlet 1422.

The system 1 may especially be configured for operating in a stationary mode and for operating in an ambulatory mode. Especially, in the stationary mode the ECMO system 200 may be connected to one or more of, especially all of, (i) a stationary power supply 1300, (ii) a stationary heater-cooler 1600, and (iii) a stationary gas supply 1500 as is depicted in the figure. Furthermore, in embodiments, in the ambulatory mode (i) the ECMO system 200 may be connected to the rechargeable power supply 300 and to the mobile gas supply 500 and (ii) the temperature control fluid inlet 1421 and the temperature control fluid outlet 1422 are closed. FIG. 1 depicts the system 1 operating in the stationary mode. Note that, in embodiments, the mobile frame 100 may support (i) the ECMO system 200 and (ii) the mobile gas supply 500, see next figures. In embodiments, the mobile gas supply 500 may comprise one or more of a gas tank (and a flow regulator), an oxygen concentrating device, etc.

The ECMO system 200 may be physically secured to the mobile frame 100. More especially, the ECMO system 200 may be supported by the mobile frame 100 around it (see also FIG. 2 and FIG. 3). The ECMO system 200 may in embodiments be a dedicated ECMO system 200. Yet, alternatively, the ECMO system 200 may be or be based on an ECMO system 200 known in the art. Hence, in embodiments, a standard ECMO system 200 already present in-house, such as in the hospital, may be used in the system 1000, and especially be arranged on the mobile frame 100.

In embodiments, the stationary system 2000 may comprise the stationary gas supply 1500, a stationary heater-cooler 1600 and the stationary power supply 1300. Especially, the ECMO system 200 may be coupled to the stationary power supply 1300. Further, in embodiments, the ECMO system 200 may be fluidically coupled to the stationary gas supply 1500. The heater cooler 1600 is especially used for heating (or cooling) blood flowing through the ECMO system 200 (i.e., external from a patient) Furthermore, the heater-cooler 1600 may be fluidically coupled to the heat exchanger 1400. The heater-cooler 1600 may especially circulate a temperature control fluid (e.g., water heated by the heater-cooler 1600) via a second side of the heat exchanger 1400 such that a first side of the heat exchanger 1400 may be heated (or cooled). The heater-cooler 1600 is discussed in more detail further below.

In embodiments, the heat exchanger 1400 may have the first side and the second side. Especially, the first side of the heat exchanger 1400 and the second side of the heat exchanger 1400 may be in thermal contact, such that the temperature of a fluid flowing through the first side may be controlled in dependence of the temperature of the fluid flowing through the second side. These sides may also be referred to as compartments or chambers. Especially, the heat exchanger 1400 comprises a temperature control fluid inlet 1421 and a temperature control fluid outlet 1422. More especially, the blood 2210 may be flowed via the temperature control fluid inlet 142, the first side of the heat exchanger 1400, and the temperature control fluid outlet 1422. Hence, in this way, the blood 2210 may especially be heated (or cooled) by the heat exchanger 1400.

The ECMO system 200 may in embodiments comprise the oxygenator 210. In embodiments, the oxygenator 210 may be configured for (i) removing carbon dioxide from the blood 2210 and (ii) providing oxygen to the blood 2210 flowing through the oxygenator 210.

As mentioned above, the system 1 may be operated in the ambulatory mode or in the stationary mode. In embodiments, in the ambulatory mode the temperature control fluid inlet 1421 and the temperature control fluid outlet 1422 are closed (especially if the ambulatory system 1000) comprises the heat exchanger 1400). In further embodiments, one of the temperature control fluid inlet 1421 or the temperature control fluid outlet 1422 are closed. The temperature control fluid in the heat exchanger 1400 may essentially still remain within the heat exchanger 1400 (even when only one of the temperature control fluid inlet 1421 or the temperature control fluid outlet 1422 are closed).

Further, in the ambulatory mode, the rechargeable power supply 300 may provide the power for (or to) the extracorporeal membrane oxygenation system 200. Especially, in the ambulatory mode, the mobile frame 100 may be moved and hence not connected to a (external) power supply. In such a scenario, the rechargeable power supply 300 may especially power the ECMO system 200. Furthermore, in embodiments, the rechargeable power supply 300 may power one or more additional power consuming elements comprised by the system 1, especially by the ECMO system 200.

It is noted that in the stationary mode, the rechargeable power supply 300 may (also) be connected to the stationary power supply 1300 to (re)charge the rechargeable power supply 300 (not shown in the figures).

Yet further, in the ambulatory mode, in embodiments, the mobile gas supply 500 may be fluidically coupled to the oxygenator 210, especially for providing an oxygen containing fluid (or oxygen comprising gas) to the blood 2210 flowing through the oxygenator 210.

In embodiments, in the stationary mode a stationary temperature control fluid supply 450 may be releasably coupled to the temperature control fluid inlet 1421. In further embodiments, a stationary temperature control fluid discharge 460 may be (releasably) coupled to the temperature control fluid outlet 1422. In embodiments, the temperature control fluid inlet 1421 and the stationary temperature control fluid supply 450 may be in open fluid connection. In further embodiments, the temperature control fluid outlet 1422 and the stationary temperature control fluid discharge 460 may be in open fluid connection.

Note that, the ambulatory system 1000 may be functionally coupled to the stationary system 2000. However, in other embodiments, the ambulatory system 1000 may (also) function independent of the stationary system 2000. For example, the stationary power supply 1300 may be wall power supply and the stationary gas supply may be a wall gas supply.

Further, in the stationary mode, a stationary power supply 1300 may be (releasably) coupled to the ECMO system 200. Especially, the stationary power supply 1300 may provide the power to the ECMO system 200. Especially, the stationary power supply 1300 may power the one or more power consuming elements of the ECMO system 200.

Yet further, in the stationary mode, a stationary gas supply 1500 may be fluidically coupled to the oxygenator 210 (for providing the oxygen containing fluid to the blood 2210 flowing through the oxygenator 210).

Further, the ambulatory system 1000 may be configured for (releasably) connecting to a stationary system 2000. Here, releasably connected refers to system 1 being configurable such that the ambulatory system 1000 may or may not be coupled to the stationary system 2000. Hence, the ambulatory system 1000 may (also) be decoupled form the stationary system 2000.

The invention may thus provide embodiments of a modular cart-in-cart system 1 (such as the ambulatory system 1000 and the stationary system 2000) comprising (consisting of) two distinct carts i.e., the stationary cart (or stationary system 2000) and the ambulatory cart (or ambulatory system 1000). The stationary cart especially carries the devices (e.g., heater-cooler 1600) not required during ambulation and may remain in the hospital (ICU) next to the patient's bed. The ambulatory cart especially carries the necessary devices during ambulation and features an extendable walking frame (or adjustable frame element 180) that ensures safe walking support for the patient. A foldable seat (mobile seat 191) allows the patient to sit and facilitates transport back to the room (ICU) in case of patient fatigue. Additionally, a safety brake system 120 prevents unintentional movement of the ambulatory cart. Devices on both carts can be (dis)connected through a multi-connector hub (docking arrangement 3000), enabling easy configuration of necessary fluids, gases, and electricity. The connection between the stationary cart and the ambulatory cart is in embodiments maintained by a foot pedal-controlled lift mechanism (not shown) that prevents accidental disconnection of the multi-connector 3000 and enables transport of both carts simultaneously. The system 1 may also features a gas switching device (gas splitter 590) that allows for convenient switching between wall-mounted gas (being an embodiment of the stationary gas supply 1500) and bottled gas (being an embodiment of the mobile gas supply 500). Further, a universal-sized shoulder brace (or body support 1100) with integrated (blood) tubing 410 that can be adjusted to the patient via e.g., Velcro closure, facilitates secure fixation of the (blood) tubing 410 in embodiments, and minimizes strain on the cannula 221.

In embodiments, the oxygenator 210 may be (releasably) connected to the one or more of the mobile gas supply 500 and the stationary gas supply 1500. In further embodiments, the ambulatory system 1000 may comprise a gas splitter 590. Especially, the gas splitter 590 may be a three-way valve. The gas splitter 590 may comprise a first inlet port 591, a second inlet port 592 and a gas outlet port 593. A gas inlet of the oxygenator 210 may be connected to the gas outlet port 593. In the depicted embodiment, the mobile gas supply 500 is connected to the first inlet port 591 and the stationary gas supply 1500 is connected to the second inlet port 592. The gas splitter 590 may be configured to fluidically connect the first inlet port 591 to a mobile gas supply outlet port 501 of the mobile gas supply 500 in the ambulatory mode and especially to connect the second inlet 592 to a stationary gas supply outlet port 1501 of the stationary gas supply 1500 in the stationary mode.

In embodiments, the system 1, especially the ambulatory system 1000, may comprise a fluid pump 400 and tubing 410. Especially, the fluid pump 400, the oxygenator 210, and the first side of the heat exchanger 1400 may be fluidically connected to each other in series via the tubing 410. In embodiments, the tubing 410 may define a first part of a fluidic circuit for the blood 2210. A second part of the fluidic circuit may especially be the circulatory system of the patient. The first part of the fluidic circuit and the second part of the fluidic circuit may especially be fluidically connected via the cannula 221 (indicated by the arrow as being at the end of the tubing 410). Especially, the blood 2210 may be flowed through the fluidic circuit. The fluid pump 400 may especially pump the blood 2210 through the fluidic circuit. Especially, the fluid pump 400 may be configured for flowing the blood 2210 through the extracorporeal membrane oxygenation system 200. The pump 400 especially may flow blood 2210 through elements of the ECMO system 2000, such as through the heat exchanger 1400 and the oxygenator 210 (and the tubing 410).

In embodiments, the mobile frame 100 is especially be configured to support a patient. These features of the system are discussed further below (see also FIG. 2 and FIG. 3).

As mentioned above, the system 1 may also comprise the stationary system 2000. In embodiments, the stationary system 2000 may comprise the heater-cooler 1600. The heater cooler 1600 may especially comprise a heater-cooler inlet 1601 configured for coupling to the stationary temperature control fluid discharge 460. In further embodiments, the heater-cooler outlet 1602 may be configured for coupling to the stationary temperature control fluid supply 450. Furthermore, the stationary system 2000 may comprise the stationary power supply 1300 for coupling to a static power line. Yet further, the stationary gas supply 1500 may be configured for connecting to one or more static gas supply lines. Note that, the static power line may in embodiments comprise a wall power supply. Especially, the one or more static gas supply lines comprises a wall gas supply.

In embodiments, the stationary system 2000 may comprise one or more gaseous supplies selected from the group of a (i) carbon dioxide-supply, (ii) a nitrogen supply, (iii) an oxygen supply, and (iv) an air supply fluidically coupled to a gas mixer for mixing the gaseous supplies to provide the gas supply 1500. The gas mixer may provide the advantage of regulating the composition of gas provided to the patient. For example, some patients (undergoing recovery) may require an enriched concentration of oxygen.

In the stationary mode, the power supply 1300 may especially be coupled to the extracorporeal membrane oxygenation system 200. Especially, the stationary gas supply 1500 may be coupled to the extracorporeal membrane oxygenation system 2000. Furthermore, the stationary temperature control fluid discharge 460 may be coupled to the temperature control fluid outlet 1422 and the stationary temperature control fluid supply 450 is coupled to the temperature control fluid inlet 1421.

The system 1 may further especially comprise a docking arrangement 3000. The docking arrangement 3000 may facilitate the docking of the stationary system 2000 and the ambulatory system 1000 such that they are functionally coupled. Hence, in embodiments, the stationary system 2000 may comprise a stationary docking part 3200. Further, in embodiments, the ambulatory system 1000 may comprise an ambulatory docking part 3100. Furthermore, the stationary docking part 3200 and the ambulatory docking part 3100 may be functionally coupled. The ambulatory docking part 3100 is especially configured for matching the stationary docking part 3200 (to functionally connect to each other).

In FIG. 1, the stationary docking part 3200 comprises a plurality of primary docking ports 3201. In the embodiment, the stationary power supply 1500 is electrically coupled to a first primary docking port 3201. A gas conduit 1550 is especially fluidically coupled to a second primary docking port 3201 and the heater-cooler inlet 1601 is coupled to a (first) third primary docking port 3201. The heater-cooler outlet 1602 is further coupled to a (second) third primary docking port 3201. Herein this is also described as the heat-cooler 1600 is functionally coupled to a (third) primary docking port 3201. A docking port 3101, 3201 may thus refer to a plurality of docking ports 3101, 3201 (together providing the functional connection).

Analogously, in embodiments, the ambulatory docking part 3100 may comprise a plurality of secondary docking ports 3101. Especially, the ECMO system 200 may be electrically coupled to a first secondary docking port 3101. In embodiments, the oxygenator 210 may be fluidically coupled to a second secondary docking port 3101. The temperature control fluid outlet 1422 may in embodiments be fluidically coupled to a (first) third secondary docking port 3101. In further embodiments, the temperature control fluid inlet 1421 may be fluidically coupled to a (second) third secondary docking port 3101. In FIG. 1, also an element with reference number 1350 is depicted. The element 1350 may e.g. refer to a transformer, or another electric element. The element 1350 may in further embodiment represent a power wire. The element 1350 may be an optional element and e.g. may in further embodiments not be present.

Hence, in the stationary mode, the stationary system 2000 may in embodiments be functionally coupled to the ambulatory system 1000 by functionally coupling the ambulatory docking part 3100 and stationary docking part 3200. Note that the ambulatory system 1000 may in embodiments (also) be decoupled from the stationary system 2000 by decoupling the ambulatory docking part 3100 and the stationary docking part 3200 (see also FIG. 3).

In embodiments, the system 1 may comprise a body support 1100 comprising a body wearable unit 1101 and a locking plate 1110. A configuration of the same in relation to the patient is provided in FIG. 2.

The body wearable unit 1101 may e.g., comprise a body wearable unit 1101 selected from the group of a shoulder strap, a head strap, a waist strap, a harness, a chest strap, an arm strap, a neck strap, and a leg strap. In further embodiments, the oxygenator 210 may comprise an oxygenator inlet 2201 and an oxygenator outlet 2202 for circulating the blood 2210). Especially, the tubing 410 may be configured for fluidically coupling the cannula 221 via the tubing 410 to the oxygenator inlet 2201 and the oxygenator outlet 2202 of the oxygenator 210 (for circulating the blood 2210 from the cannula 221 through the oxygenator 210. In embodiments, the locking plate 1110 may be configured for immobilizing the tubing 410 (hence, especially the cannula 221), via the tubing 410. The locking plate 1110 especially immobilizes the tubing 410, especially by fixating the tubing at the holding plate 1110.

In further embodiments, the cannula 221 may comprise one or more cannula inserts (or lumens) 2211. Especially, the one or more cannula inserts 2211 are configured to be inserted in an artery and/or a vein.

FIG. 2 schematically depicts some further aspects of the system 1 and further also the use of said system 1 supporting a patient. In embodiments, the mobile frame 100 may be configured to support a patient functionally connected to the extracorporeal membrane oxygenation system 200 at a first side of the mobile frame 100. In the figure, the first side may especially be the side of the mobile frame 100 directly in front of the patient. Especially, the patient may lean on the first side of the mobile frame 100. In further embodiments, the mobile frame 100 may comprise an adjustable frame element 180 configured for extending from the first side of the mobile frame 100 for supporting the patient. In further embodiments, the adjustable frame element 180 may be configured for extending along (side of) one or two sides of the patient, for supporting the patient while walking. In the figure, it can be observed that the mobile frame 100 comprises two adjustable frame elements 180 extending to the lateral sides of the patient. Especially, the patient may be support by the mobile frame 100 (i.e., the first side of the mobile frame 100) and the adjustable frame elements 180 on the lateral sides of the patient. This way the patient can move, such as walk, with the ambulatory system 2000, and exercise.

In further embodiments, the adjustable frame element 180 may comprise a seating support 190 configured for supporting the patient while sitting. Especially, the patient may be supported by the seating support 190 on at least one lateral side of the patient. Furthermore, in embodiments, the seating support 190 may comprise a mobile seat 191. The figure depicts the patient standing, however, the patient may (also) be seated on the mobile seat 191. The mobile frame 100 may in embodiments be configured for moving the patient supported by the seating support 190 in a seated position relative to a remainder of the frame 100. In embodiments, the patient may be supported by the seating support 190. Especially, the patient may be movable (or mobile). Herein, the term “move” in relation to movement of the patient may refer to moving the patient, wherein the patient walks or does another physical action (the mobile frame element 180 may e.g., in further embodiment comprise a bike attachment like configurations on which the patient may ride to exercise while moving). It may further refer to a movement of the ambulatory system 1 together with the patient (such as sitting or standing on the frame 100).

Note that in embodiments, at least part of the adjustable frame element 180 may be removable from the frame 100 for moving the patient to and from the bed to a location on the first side of the frame 100.

As mentioned above, it can be observed that the cannula tubing 410 may fluidically connect the ECMO system 200 (especially the oxygenator 210) to the cannula 221. The body wearable unit 1101 may especially be strapped onto the patient. Further, in embodiments, the locking plate 1110 may especially secure the tubing 410 such that there is no relative motion of the tubing 410 between the cannula 221 and locking plate 1110. Hence, in this way, the patient may be moved without dislodging the cannula inserts 2211.

FIG. 3 depicts an embodiment of the system 1 illustrating the docking of the ambulatory system 1000 and the stationary system 2000. The system 1 may be configured for (releasably) connecting the ambulatory system 1000 to the stationary system 2000. Especially, the stationary system 2000 may be movable with the ambulatory system 1000. At the left hand side, the ambulatory system 1000 and the stationary system 2000 are not functionally connected. At the right hand side, the systems 1000, 2000 are functionally coupled among others via the docking arrangement 3000. The ambulatory system 1000 especially comprises wheels for moving the ambulatory system 1000, especially the frame 100.

In embodiments, the stationary system 2000 may comprise stationary system wheels for moving the stationary system 2000. Especially, the mobile frame 100 may comprise a lift mechanism to lift the stationary system 2000. In embodiments, in an operational mode the stationary system 2000 may be lifted and physically secured to the mobile frame 100 (wherein the stationary system wheels are disabled).

In further embodiments, the mobile frame 100 may comprise a braking system 120 (very schematically depicted in FIG. 1). Especially, the braking system 120 comprises a braking lever 121 (for unlocking the brake), wherein the mobile frame 100 is configured immobile by default. The braking lever 121 may, e.g., be configured at a frame element 180 held by the patient during moving.

Further, the ambulatory system 1000 depicted in FIGS. 2 and 3 comprise a medical element 1010, such as an intra-venous drip, a heart rate monitor, a perfuser pump, emergency pump drive, an emergency kit, etc. Furthermore, the medical element 1010 may be functionally coupled to the mobile frame 100.

The term “plurality” refers to two or more. Furthermore, the terms “a plurality of” and “a number of” may be used interchangeably.

The terms “substantially” or “essentially” herein, and similar terms, will be understood by the person skilled in the art. The terms “substantially” or “essentially” may also include embodiments with “entirely”, “completely”, “all”, etc. Hence, in embodiments the adjective substantially or essentially may also be removed. Where applicable, the term “substantially” or the term “essentially” may also relate to 90% or higher, such as 95% or higher, especially 99% or higher, even more especially 99.5% or higher, including 100%. Moreover, the terms “about” and “approximately” may also relate to 90% or higher, such as 95% or higher, especially 99% or higher, even more especially 99.5% or higher, including 100%. For numerical values it is to be understood that the terms “substantially”, “essentially”, “about”, and “approximately” may also relate to the range of 90%-110%, such as 95%-105%, especially 99%-101% of the values(s) it refers to.

The term “comprise” also includes embodiments wherein the term “comprises” means “consists of”.

The term “and/or” especially relates to one or more of the items mentioned before and after “and/or”. For instance, a phrase “item 1 and/or item 2” and similar phrases may relate to one or more of item 1 and item 2. The term “comprising” may in an embodiment refer to “consisting of” but may in another embodiment also refer to “containing at least the defined species and optionally one or more other species”.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The devices, apparatus, or systems may herein amongst others be described during operation. As will be clear to the person skilled in the art, the invention is not limited to methods of operation, or devices, apparatus, or systems in operation.

The term “further embodiment” and similar terms may refer to an embodiment comprising the features of the previously discussed embodiment but may also refer to an alternative embodiment.

It should be noted that the above-mentioned embodiments illustrate rather than limit the invention, and that those skilled in the art will be able to design many alternative embodiments without departing from the scope of the appended claims.

In the claims, any reference signs placed between parentheses shall not be construed as limiting the claim.

Use of the verb “to comprise” and its conjugations does not exclude the presence of elements or steps other than those stated in a claim. Unless the context clearly requires otherwise, throughout the description and the claims, the words “comprise”, “comprising”, “include”, “including”, “contain”, “containing” and the like are to be construed in an inclusive sense as opposed to an exclusive or exhaustive sense; that is to say, in the sense of “including, but not limited to”.

The article “a” or “an” preceding an element does not exclude the presence of a plurality of such elements.

The invention may be implemented by means of hardware comprising several distinct elements, and by means of a suitably programmed computer. In a device claim, or an apparatus claim, or a system claim, enumerating several means, several of these means may be embodied by one and the same item of hardware. The mere fact that certain measures are recited in mutually different dependent claims does not indicate that a combination of these measures cannot be used to advantage.

The invention also provides a control system that may control the device, apparatus, or system, or that may execute the herein described method or process. Yet further, the invention also provides a computer program product, when running on a computer which is functionally coupled to or comprised by the device, apparatus, or system, controls one or more controllable elements of such device, apparatus, or system.

The invention further applies to a device, apparatus, or system comprising one or more of the characterizing features described in the description and/or shown in the attached drawings. The invention further pertains to a method or process comprising one or more of the characterizing features described in the description and/or shown in the attached drawings. Moreover, if a method or an embodiment of the method is described being executed in a device, apparatus, or system, it will be understood that the device, apparatus, or system is suitable for or configured for (executing) the method or the embodiment of the method, respectively.

The various aspects discussed in this patent can be combined in order to provide additional advantages. Further, the person skilled in the art will understand that embodiments can be combined, and that also more than two embodiments can be combined. Furthermore, some of the features can form the basis for one or more divisional applications.

1. A system to provide life support, comprising an ambulatory system, the ambulatory system comprising an extracorporeal membrane oxygenation system, a mobile gas supply, and a mobile frame, wherein:
the extracorporeal membrane oxygenation system comprises (i) a rechargeable power supply, (ii) an oxygenator, and (iii) a heat exchanger for controlling a temperature of blood flowing through the extracorporeal membrane oxygenation system, wherein the heat exchanger comprises a temperature control fluid inlet and a temperature control fluid outlet,
the system is configured for operating in an ambulatory mode and for operating in a stationary mode;
wherein in the ambulatory mode:
(i) the temperature control fluid inlet and the temperature control fluid outlet are closed,
(ii) the rechargeable power supply provides the power for the extracorporeal membrane oxygenation system, and
(iii) the mobile gas supply is fluidically coupled to the oxygenator; and
wherein in the stationary mode:
(i) a stationary temperature control fluid supply is coupled to the temperature control fluid inlet and a stationary temperature control fluid discharge is coupled to the temperature control fluid outlet,
(ii) a stationary power supply is coupled to the extracorporeal membrane oxygenation system, wherein the stationary power supply provides the power to the extracorporeal membrane oxygenation system, and
(iii) a stationary gas supply is fluidically coupled to the oxygenator.

2. The system according to claim 1, wherein the ambulatory system comprises a gas splitter, wherein the gas splitter comprises a first inlet port, a second inlet port and a gas outlet port, wherein:
the oxygenator is connected to the gas outlet port;
the mobile gas supply is connected to the first inlet port and the stationary gas supply is connected to the second inlet port; and
the gas splitter is configured to fluidically connect (i) the first inlet port to the mobile gas supply in the ambulatory mode and (ii) the second inlet to the stationary gas supply in the stationary mode.

3. The system according to claim 2, wherein the mobile gas supply comprises one or more of a gas tank, a pony bottle, and an oxygen concentrating device, wherein the stationary gas supply is configured for coupling to a static gas supply line.

4. The system according to claim 1, wherein the extracorporeal membrane oxygenation system comprises a fluid pump and tubing, wherein the fluid pump, the oxygenator, the heat exchanger, and the tubing define a first part of a fluidic circuit for the blood, wherein the fluid pump is configured for flowing the blood through the extracorporeal membrane oxygenation system.

5. The system according to claim 4, wherein the system comprises a body support comprising a body wearable unit, and a locking plate, wherein:
the body wearable unit is selected from the group of a shoulder strap, a head strap, a waist strap, a harness, a chest strap, an arm strap, a neck strap, and a leg strap;
the locking plate is configured for immobilizing the tubing, wherein the tubing fluidically connects the extracorporeal membrane oxygenation system to a cannula.

6. The system according to claim 5, wherein the cannula comprises one or more cannula inserts, wherein the one or more cannula inserts are configured to be inserted in an artery and/or a vein.

7. The system according to claim 1, wherein the extracorporeal membrane oxygenation system is supported by the mobile frame.

8. The system according to claim 1, wherein the mobile frame is configured to support a patient at a first side of the mobile frame, wherein the mobile frame comprises an adjustable frame element configured for extending from the first side of the mobile frame for supporting the patient.

9. The system according to claim 8, wherein the adjustable frame element is configured for extending along one or two sides of the patient, for supporting the patient while walking.

10. The system according to claim 8, wherein the adjustable frame element comprises a seating support configured for supporting the patient while sitting.

11. The system according to the claim 10, wherein the seating support comprises a mobile seat configured for moving the patient in a seated position relative to a remainder of the frame.

12. The system according to claim 9, wherein at least part of the adjustable frame element is removable from the frame for moving the patient to and from the bed to a location on the first side of the mobile frame.

13. The system according to claim 1, wherein the ambulatory system comprises a medical element selected from the group of an intra-venous drip, a heart rate monitor, a perfuser pump, an emergency pump drive and an emergency kit, wherein the medical element is functionally coupled to the mobile frame.

14. The system according to claim 1, wherein the mobile frame comprises a braking system, wherein the braking system comprises a braking lever, wherein the mobile frame is configured immobile by default.

15. The system according to claim 1, wherein the system further comprises a stationary system, wherein the stationary system comprises the heater-cooler comprising a heater-cooler inlet configured for coupling to the stationary temperature control fluid discharge and a heater-cooler outlet configured for coupling to the stationary temperature control fluid supply, wherein the stationary power supply configured for coupling to a static power line, wherein the stationary gas supply configured for connecting to one or more static gas supply lines.

16. The system according to claim 15, wherein the system comprises a docking arrangement, wherein the docking arrangement comprises an ambulatory docking part and a stationary docking part, wherein:
the stationary docking part comprises a plurality of primary docking ports, wherein the stationary power supply is electrically coupled to a first primary docking port, wherein the gas conduit is fluidically coupled to a second primary docking port, and wherein the heater-cooler inlet and the heater-cooler outlet are fluidically coupled to a third primary docking port;
the ambulatory docking part comprises a plurality of secondary docking ports wherein the extracorporeal membrane oxygenation system is functionally coupled to the secondary docking ports;
in the stationary mode the ambulatory docking part and the stationary docking part are coupled, wherein the extracorporeal membrane oxygenation system is fluidically coupled to the stationary gas supply, wherein the extracorporeal membrane oxygenation system is coupled to the stationary power supply, and wherein the temperature control fluid inlet is coupled to the heater-cooler outlet and the temperature control fluid outlet is coupled to the heater-cooler inlet.

17. The system according to claim 15, wherein the system is configured for connecting the ambulatory system to the stationary system, wherein the stationary system is movable with the ambulatory system.

18. The system according to claim 2, wherein the extracorporeal membrane oxygenation system comprises a fluid pump and tubing, wherein the fluid pump, the oxygenator, the heat exchanger, and the tubing define a first part of a fluidic circuit for the blood, wherein the fluid pump is configured for flowing the blood through the extracorporeal membrane oxygenation system the adjustable frame element comprises a seating support configured for supporting the patient while sitting.

19. The system according to the claim 2, wherein T the extracorporeal membrane oxygenation system is supported by the mobile frame.

20. The system according to claim 2, wherein the mobile frame is configured to support a patient at a first side of the mobile frame, wherein the mobile frame comprises an adjustable frame element configured for extending from the first side of the mobile frame for supporting the patient.
